(19) United States
(12) Patent Application Publication
(10) Pub. No.: US 2012/0029177 A1
(43) Pub. Date: Feb. 2, 2012

(54) HETEROCYCLE-AMINO ACID DERIVATIVES FOR TARGETING CANCER TISSUE AND RADIOACTIVE OR NON-RADIOACTIVE LABELED COMPOUNDS THEREOF

(75) Inventors: Jae Min Jeong, Seoul (KR); Dinesh Shetty, Seoul (KR); Dong Soo Lee, Seoul (KR); June Key Chung, Seoul (KR); Myung Chul Lee, Seoul (KR)

(73) Assignee: SNU R&DB FOUNDATION, Seoul (KR)

(21) Appl. No.: 13/262,043

(22) PCT Filed: Mar. 31, 2010
(86) PCT No.: PCT/KR10/01981

§ 371 Date: Sep. 29, 2011

(30) Foreign Application Priority Data
Apr. 3, 2009 (KR) 10-2009-0029126

(51) Int. Cl. C07D 255/02; C07F 1/08; C07F 5/00; C07F 13/00; C07F 15/04; C07F 15/06; C07F 17/00; C07F 11/00; C07D 257/02; C07F 15/02

(52) U.S. Cl. 534/16; 540/474; 540/465

(57) ABSTRACT

The present invention relates to novel amino acid derivatives containing heterocyclic chelating residues thereof; radioactive or nonradioactive metal complexes thereof; methods for preparation thereof; and apyrogenic and sterile preparative kits of the composition for targeting cancer cells.

The compounds of the present invention can easily be taken up to cancer cells as they contain amino acid residues thereof; radioactive or nonradioactive metals can be labeled easily as they contain heterocyclic chelating residues thereof; cancer lesion can be imaged easily by targeting using the present invention.

### TECHNICAL FIELD

The present invention relates to novel cancer targeting agents for obtaining magnetic resonance images (MRI) or radionuclide images.

### BACKGROUND ART

Amino acid transporters are highly expressed in the tissues with high protein synthesis rate and transport amino acids into the cells as the protein precursors. As pancreas synthesizes digestive enzymes and as cancer tissues are rapidly proliferating, they are typical examples of high protein synthesis tissues. As cancer tissues take up amino acid rapidly; nuclear imaging, MRI, or neutron therapy can be performed using radio labeled, magnetic metal labeled, or boron conjugated amino acids, respectively.

Although almost all solid tumors can be imaged using radiolabeled amino acids, brain tumor, head and neck cancers, and lung cancers were reported as typical examples. The most widely used radiolabeled amino acid for nuclear medicine imaging is [¹¹C]methionine. (Chung J-K, Kim Y K, Kim S-K, et al. Usefulness of ¹¹C-methionine PET in the evaluation of brain lesions that are hypo- or isometabolic on ¹⁸F-FDG PET, Eur J Nucl Med 2002; 29:176-182; Fujiwara T, Matsuzawa T, Kubota K, et al. Relationship between type of primary lung cancer and carbon-11-L-methionine uptake with positron emission tomography. J Nucl Med 1989; 30:33-37; Leskinen-Kallio S, Nagren K, Lehikoinen P, Ruotsalainen U, Tearaes M, Joensuu H. Carbon-11-methionine and PET is an effective method to image head and neck cancer. J Nucl Med 1992; 33:691-695).

Although [¹¹C]methionine is an excellent radiopharmaceutical for imaging brain tumor, lung cancer, and head and neck cancer, it is not adequate for obtaining many patients' images or transporting to distant places due to its short half life (20 min).

Thus, ¹⁸F-labeled amino acid derivatives were developed as it has longer half life (110 min). [¹⁸F]fluoroethyltyrosine (FET) and [¹⁸F]FACBC are most famous among them. (Weber W, Wester H-J, Grosu A, et al. O-(2-([¹⁸F]Fluoroethyl)-L-tyrosine and L-[methyl-11C-methionine uptake in brain tumours: initial results of a comparative study. Eur J Nucl Med 2000; 27:542-549; Tang G, Wang M, Tang X, Luo L, Gan M. Fully automated synthesis module for preparation of S-(2-[¹⁸F]fluoroethyl)-L-methionine by direct nucleophilic exchange on a quaternary 4-aminopyridinium resin. Nucl Med Biol 2003; 30:509-512; US Patent US2007/0082879 A1, Goodman M M, Apr. 12, 2007, Imaging agents. Assignee Emory University, Atlanta, Ga.; Martarello L, McConathy J, Camp V M, et al. Synthesis of syn- and anti-1-amino-3-[¹⁸F]fluoromethyl-cyclobutane-1-carboxylic acid (FMACBC), potential PET ligands for tumor detection. J Med Chem 2002; 45:2250-2259). Although ¹⁸F can be distributed after production due to its relatively longer half life, it still has problems, because it requires expensive cyclotron system, complicate synthesis procedure, and long synthesis time.

### DISCLOSURE OF THE INVENTION

The object of the present invention is to synthesize novel amino acid derivatives showing excellent characteristics for cancer imaging, and to provide precursors for easy radio labeling to synthesize labeled compounds.

The object of the present invention is to provide heterocycle-amino acid derivatives labeled with radioactive or nonradioactive metals selected from Cr, Fe, Co, Ni, Cu, Ga, Sr, Y, Zr, Mo, Tc, Ru, Rh, Pd, Cd, In, Sn, Ba, La, Sm, Gd, Dy, Ho, Lu, Re, Ir, Pb, and Bi. ⁶⁸Ga is the most ideal element among the above metals.

Moreover, the object of the present invention comprises the synthesis method of precursor and radioactive or nonradioactive metal labeled precursor.

Another object of the present invention comprises pharmaceutically acceptable kit which contains above described precursors to facilitate radiolabeling. In detail, buffer solution is added to the above precursors, and then the solution is dispensed into pharmaceutically acceptable vials and sealed. The vials can be used as is or can be used when required after refrigeration, frozen, or lyophilization.

### Means for Solving the Problems

The present invention provides the following compounds, complex comprising these compounds, and a kit:

[1] a compound of Formula (1) or a pharmaceutically acceptable salt thereof:

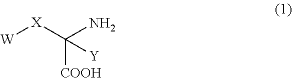

wherein W is

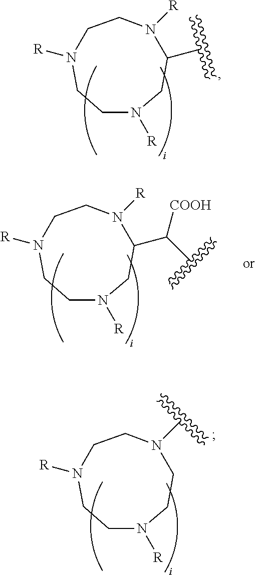

R is independently H, C₁ to C₆ alkyl, or —(CH₂)ₐCOOH; a and i are independently integer 1 to 6; X is repeatedly or non-repeatedly linked structure composed of 1 to 6 residue(s) of one or more selected from the group consisting of —CH₂—, —NH—, —O—, —S—, —CS—, and —CO—; and Y is H or a methyl residue;
[2] the compound or a pharmaceutically acceptable salt thereof according to [1], wherein W is

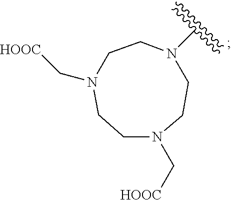

### X and

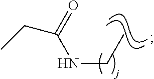

j is an integer 1 to 6;
[3] the compound or a pharmaceutically acceptable salt thereof according to [1] or [2], wherein W is

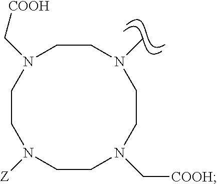

### X is

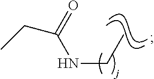

j is an integer 1 to 6; and Z is H or —CH₂COOH;
[4] the compound or a pharmaceutically acceptable salt thereof according to any one of [1] to [3], wherein X is CH₂ₖ; and k is an integer 1 to 6;
[5] the compound or a pharmaceutically acceptable salt thereof according to any one of [1] to [4], wherein amino acid arrangement is L type isomer;
[6] the compound or a pharmaceutically acceptable salt thereof according to any one of [1] to [4], wherein amino acid arrangement is D type isomer;
[7] a complex of radioactive or non-radioactive metals with the compound or a pharmaceutically acceptable salt thereof according to any one of [1] to [6];
[8] the complex according to [7], wherein radioactive or non-radioactive metals are selected from the group consisting of Cr, Fe, Co, Ni, Cu, Ga, Sr, Y, Zr, Mo, Tc, Ru, Rh, Pd, Cd, In, Sn, Ba, La, Sm, Gd, Dy, Ho, Lu, Re, Ir, Pb, and Bi;
[9] the complex according to [7], wherein radioactive metals are selected from the group consisting of ¹¹¹In, ⁶⁸Ga, ⁶⁷Ga, ⁶⁰Cu, ⁶¹Cu, ⁶²Cu, ⁶⁴Cu, ⁶⁷Cu, ⁸⁵Y, ⁸⁶Y, ⁸⁷Y, ⁹⁰Y, ¹⁷⁷Lu, ^{117m}Sn, ¹⁰³Pd, and ¹⁶⁶Ho;
[10] a kit for the preparation of a sterile non-pyrogenic sealed vial of solution, frozen or lyophilized state containing 1 ng˜100 mg of the compound or a pharmaceutically acceptable salt thereof according to any one of [1] to [6];

### Effects of the Present Invention

Heterocycle-amino acid derivatives of the present invention are useful for cancer diagnosis and therapy because they have excellent effect of targeting cancer tissues and have high labeling efficiency and rapid labeling reaction with radioactive or non-radioactive metals. Especially, they can be extensively distributed, because they can be easily used in middle or small size hospitals without special facilities and personnel by supplied as pharmaceutical kit form which can be labeled with simple procedure.

As the heterocycle-amino acid derivatives of the present invention can be prepared straightforwardly by simple mixing or simple mixing with heating within short time (10 min), they have advantages over ¹⁸F or ¹¹C labeled amino acid derivatives which should be prepared by complicate organic synthesis. In addition, radiometal such as ⁶⁸Ga has advantages of low price and producible from convenient generator.

### DESCRIPTION OF THE PRESENT INVENTION

The present invention relates to heterocycle-amino acid derivatives described as Formula (1) or their pharmaceutically acceptable salts and their complexes with radioactive or non-radioactive metals.

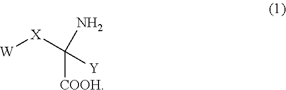

### W is

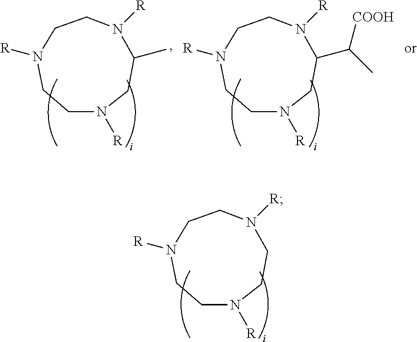

R is independently H, C₁-C₆ alkyl, or —(CH₂)ₐCOOH; a and i are independently integer 1 to 6; X is repeatedly or non-repeatedly linked structure composed of 1 to 6 residue(s) of one or more selected from the group consisting of —CH₂—, —NH—, —O—, —S—, —CS—, and —CO—; and Y is H or a methyl residue.

The heterocycle residues of the above compounds can form complexes with radioactive or non-radioactive metals and form amino acids containing metal. These amino acids can be accumulated in cancer cells by amino acid transporters highly expressed in cancer cells. Thus, if the metal is magnetic then MRI can be performed, and if the metal is radioactive, nuclear imaging or radionuclide therapy can be performed.

Moreover, ⁶⁸Ga with a half-life of 68 min can be used to solve the problems of conventional ¹¹C or ¹⁸F labeled amino acids. The highest advantage of ⁶⁸Ga is that it can be easily produced from a generator which is relatively very cheaper than cyclotron. In addition, it can be put to practical use very easily because of very short labeling time. (Breeman W A, Verbruggen A M. The ⁶⁸Ge/⁶⁸Ga generator has high potential, but when can we use ⁶⁸Ga-labelled tracers in clinical routine? Eur J Nucl Med Mol Imaging. 2007; 34:978.981; Zhernosekkov K P, Filosofov D V, Baum R P, et al. Processing of generator-produced ⁶⁸Ga for medical application. J Nucl Med 2007; 48:1741-1748) Thus, the ⁶⁸Ga labeled amino acids can be used as economically and easily labeled positron emitting radiopharmaceuticals.

Heterocycle-amino acids that can be labeled with ⁶⁸Ga also can be labeled with other radioactive or non-radioactive metals. For example, ¹¹¹In can be used for single photon emission computed tomography (SPECT) which is cheaper than PET. (Onthank D C, Liu S, Silva P J, Barrett J A, Harris T D, Robinson S P, Edwards D S, ⁹⁰Y and ¹¹¹In Complexes of a DOTA-Conjugated Integrin rva3 Receptor Antagonist: Different but Biologically Equivalent. Bioconjugate Chem 2004, 15:235-241), radioactive coppers can be used for obtaining long time images or cancer therapy, although they are produced by an expensive cyclotron, (Sprague J E, Peng Y, Framengo A L, Woodin K S, Southwick E A, Weisman G R, Wong E H, Golen J A, Rheingold A L, Anderson C J. Synthesis, Characterization and In Vivo Studies of Cu(II)-64-Labeled Cross-Bridged Tetraazamacrocycle-amide Complexes as Models of Peptide Conjugate Imaging Agents. Bioconjugate Chem 2007, 50:2527-2535.) and radioactive yttrium, lutetium, and holmium can be used for therapy with high effect (Cremonesil M, Ferraril M, Bodei L, Tosil G, Paganelli G. Dosimetry in Peptide Radionuclide Receptor Therapy: A Review. J Nucl Med 2006, 47:1467-1475)

For the agents of the present invention to be taken up by cancer cells via amino acid transporters, R groups of the amino acids should be maintained as small. In the present invention, R groups should have chelate because R group should be labeled with metallic radionuclides such as ⁶⁸Ga. The basic compounds of the present invention was designed by conjugating amino and carboxyl groups in short distance to the famous heterocyclic agents for ⁶⁸Ga labeling such as 1,4,7-triazacyclononane-1,4,7-triacetic acid (NOTA) or 1,4,7,10-tetraazacyclododecane-1,4,7,10-tetraacetic acid (DOTA). If the radionuclides were ^{99m}Tc or ¹⁸⁸Re, [^{99m}Tc(CO)₃]⁺ or N₂S₂ might give more efficient and stable compounds. (Liu Y, Pak J K, Schmutz P, et al. Amino acids labeled with [^{99m}Tc(CO)₃]⁺ and recognized by the L-type amino acid transporter LAT1. J Am Chem Soc 2006; 128:15996-15997; US Patent 2005/0192458 A1, Goodman M M, McConathy J, Tumor imaging compounds. Pub date Sep. 1, 2005)

The kits for preparation of non-pyrogenic sterile radiometal labeled agents of the present invention are composed of heterocycle-amino acid derivatives and adequate buffer solution dispensed into sterile vials, frozen or lyophilized, and sealed. The kits can be stored and used when they are required.

### BRIEF EXPLANATION OF DRAWINGS

FIG. 1a shows the U87MG cell uptake of ⁶⁸Ga-DOTA-ala, ⁶⁸Ga-DOTA, ⁶⁸Ga-NOTA-ala, and ⁶⁸Ga-NOTA.

FIG. 1b shows the CT-26 cell uptake of ⁶⁸Ga-DO2A-ala, ⁶⁸Ga-DO2A, ⁶⁸Ga-DO3A-ala, and ⁶⁸Ga-DO3A.

FIG. 1c shows the Hep3B cell uptake of ⁶⁸Ga-NOTA-homoser, ⁶⁸Ga-NOTA-lys, ⁶⁸Ga-NOTA-ala, and ⁶⁸Ga-NOTA.

FIG. 2 shows the PET images of ⁶⁸Ga-NOTA-ala, ⁶⁸Ga-DOTA-ala, ⁶⁸Ga-DO2A-ala, and ⁶⁸Ga-DO3A-ala in SNU-C4 xenografted nude mice.

### EXAMPLES

The following examples are given to illustrate the present invention. It should be understood, however, that the invention is not to be limited to the specific conditions or details described in these examples.

¹H-NMR (300 MHz) spectra were obtained by AL 300 FT (Jeol Co.). Chemical shifts were represented by the shift to down field compared to tetramethylsilane. Optical rotation was measured using P-1020 polarimeter (Jasco). HPLC was performed using Agilent 1100 series equipped with XTerra 10 μm RP18 (10×250 mm) column. Solvent A was 0.01% trifluoroacetic acid (TFA) aqueous solution and Solvent B was acetonitrile. Flow rates were 1 mL/min for analysis and 5 mL/min for preparation. Mass spectra (ESI-MS) were obtained using Waters ESI ion-trap spectrometer.

DOTA and NOTA were purchased from ChemTech Co. (France), and DO2AtBu and DO3AtBu were purchased from Macrocyclics (U.S.A.). Beta-serine lactone was purchased from TCI (Japan) and acetonitrile for HPLC was purchased from Fischer Scientific Ltd (U.S.A.). Other reagents without description were purchased from Sigma-Aldrich-Fluka (U.S.A.).

2-tert-Butoxycarbonylamino-3-methanesulfonyloxy-propionic acid methyl ester was synthesized as follows. N-tert-butyl-L-serine methyl ester (2 g, 9.1 mmol) and triethylamine (1 g, 10 mmol) were added in a flask and dissolved in 50 mL methylenechloride. The solution was cooled with ice and methansulfonylchloride (1.15 g, 10 mmol) was added slowly with stirring. The reaction mixture was washed by 25 mL water and organic layer was recovered, dehydrated with sodium sulfate, and dried under reduced pressure to give colorless oil (2.2 g, 8.5 mmol). Dimethyl form amide (DMF, 60 mL) and sodium azide (1.4 g, 21 mmol) were add and reacted for 30 min at 50° C. with stirring. Cold water (200 mL) was added and was extracted using 60 mL ethylacetate 2 times. The organic layers were pooled, dehydrated by sodium sulfate, and dried under reduced pressure to give yellowish oil. The product was purified by silica gel column using ethylacetate:hexane (1:5) solution and obtained colorless oil (1 g, 56%). 10% Pd—C (60 mg) and absolute ethanol (10 mL) were added and reacted for 1 hr with mixing under 1 atm hydrogen at room temperature. The catalyzer was removed by celite filter and the filter was washed by ethanol. The filtrate was evaporated under reduced pressure to give colorless oil (700 mg, 79%) as the final product.

¹H NMR (300 MHz, CD CDCl₃, ppm): δ 1.44 (9H, s), 3.72 (2H, br), 3.74 (3H, s), 4.45 (1H, br), 5.85 (1H, br, —NH).

¹³C NMR (80 MHz, CDCl₃, ppm): δ 28.2, 42.5, 52.7, 53.9, 80.2, 155.6, 171.2.

Mass spectrum (ESI+), m/z 219 (M+H)⁺.

[α]_{D}¹⁹=−18 (c=0.5, EtOH).

### Example 1

### Synthesis of DO2A-ala

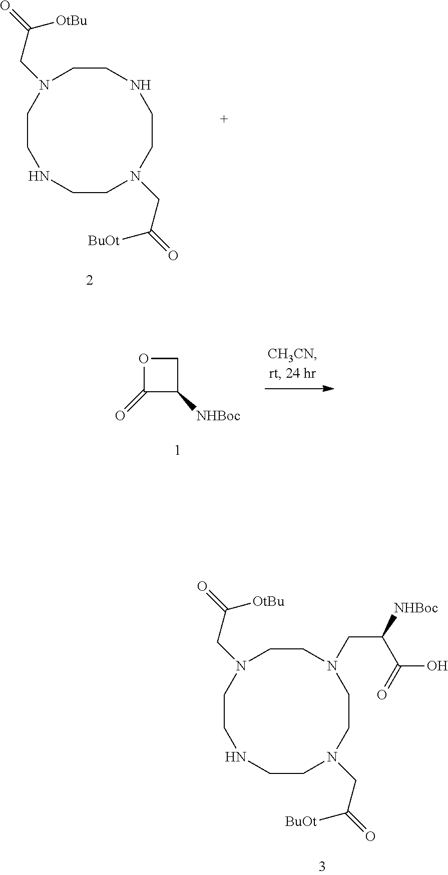

To a solution of DO2tBu (1,7-bis-tert-butoxycarbonylmethyl-1,4,7,10-tetraaza-cyclodode-1-yl)-acetic acid tert-butyl ester (0.2 g, 0.49 mmol) in anhydrous acetonitrile (5 mL), N-(tert-butoxycarbonyl)-L-serine-β-lactone (0.1 g, 0.29 mmol) solution in anhydrous acetonitrile (2 mL) was slowly added under nitrogen and stirred for 24 hr at room temperature. White solid (0.13 g, 89%) formed after reaction was obtained by filtration and washed with acetonitrile. The purity of the product was checked by TLC.

¹H NMR (300 MHz, CDCl₃, ppm): δ 5.81 (1H, —NH—), 4.05 (1H, br), 3.35 (4H, s), 3.15 (2H, br), 2.62-3.07 (16H, br), 1.41-1.45 (27H, ss).

¹³C NMR (80 MHz, CDCl₃, ppm): δ 176.2 (CO), 170.7 (CO), 155.2 (CO), 81.3, 78.6, 58.8, 55.8 (—NCH₂CH—), 54.2 (—CH₂CH—), 45.4, 28.4, 28.1.

Mass spectrum (ESI+), m/z 588.2 (M+H)⁺.

[α]_{D}^{20.4}+30.3 (c=0.5, CHCl₃)

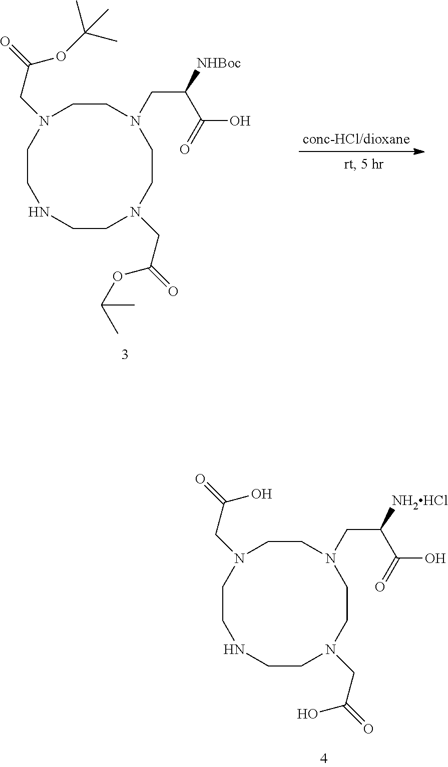

To a solution of the above obtained compound (0.13 g) in 1,4-dioxane (3 mL), conc-HCl (0.6 mL) was slowly added and stirred for 5 hr at room temperature. The reaction mixture was purified using HPLC and pure compound was obtained as HCl salt at retention time 2.6 min (80 mg, 90%).

¹H NMR (300 MHz, D₂O, ppm): δ 4.15 (1H, br), 3.49 (2H, br), 3.20 (4H, s), 2.45-3.0 (16H, br).

¹³C NMR (80 MHz, D₂O, ppm): δ 174.7 (CO), 171.2 (CO), 52.9, 50.9 (—NCH₂CH—), 49.6 (—NCH₂CH—), 47.1, 42.9.

Mass spectrum (ESI+), m/z 588.2 (M+H)⁺, HRMS, observed mass m/z 376.2198.

[α]_{D}^{20.4}=+21.4 (c=0.38, CH₃OH)

### Example 2

### Synthesis of DO3A-ala

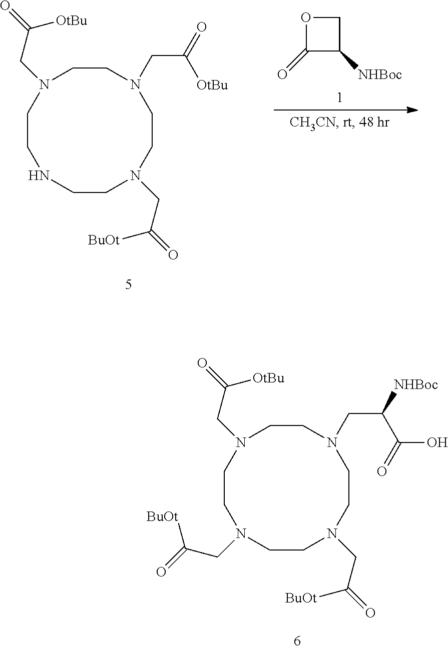

To a solution of DO3tBu (1,4,7-tris-tert-butoxycarbonylmethyl-1,4,7,10-tetraaza-cyclodode-1-yl)-acetic acid tert-butyl ester (0.2 g, 0.39 mmol) in anhydrous acetonitrile (5 mL), N-(tert-butoxycarbonyl)-L-serine-β-lactone (0.087 g, 0.47 mmol) solution in anhydrous acetonitrile (2 mL) was slowly added under nitrogen and stirred for 48 hr at room temperature. The reaction mixture was purified using HPLC and the product peak at 9 min was harvested.

¹H NMR (300 MHz, CDCl₃, ppm): δ 6.08 (1H, —NH—), 4.03-4.10 (1H, m), 3.65-3.70 (1H, t), 3.15-3.40 (12H, br), 2.72-2.82 (8H, br), 1.42-1.45 (27H, ss).

¹³C NMR (80 MHz, CDCl₃, ppm): δ 171.8 (CO), 170.5 (CO), 156.2 (CO), 81.5, 79.5, 56.2, 54.8 (—NCH2CH—), 53.6 (—NCH2CH—), 49.7, 28.4, 28.1.

Mass spectrum (ESI+), m/z 702.4 (M+H)⁺.

[α]_{D}^{20.4}=+30.3 (c=0.5, CHCl₃)

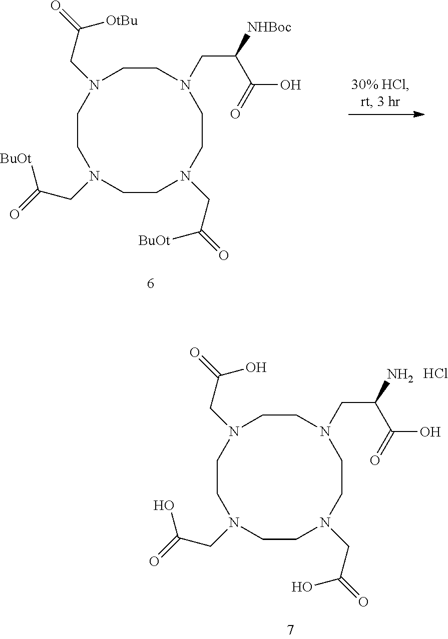

The above obtained compound was dissolved in 30% HCl and stirred for 3 hr at room temperature. The reaction mixture was purified by HPLC and pure product was obtained as HCl salt at 2.7 min peak (50 mg, 83%).

¹H NMR (300 MHz, D₂O, ppm): δ 3.98 (6H, s), 3.67 (1H, br), 2.85-3.38 (16H, br), 2.45-2.53 (4H, br).

¹³C NMR (80 MHz, D₂O, ppm): δ 170.7 (CO), 56.7, 55.2, 53.5 (—NCH₂CH—), 53.2 (—NCH₂CH—), 50.8, 50.1.

Mass spectrum (ESI+), m/z 434.2 (M+H)⁺, HRMS, observed mass m/z 376.2245.

[α]_{D}^{20.4}=+13.6 (c=0.38, CH₃OH)

### Example 3

### Synthesis of NOTA-ala

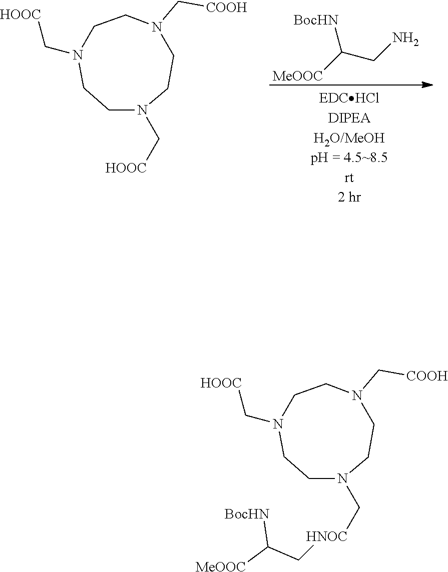

Protected aminoalanine (0.020 g, 0.09 mmol) solution in methanol (0.2 mL) was slowly added to 1.5 mL aqueous solution of 0.05 g (0.16 mmol) NOTA. The mixture was cooled by ice and pH was adjusted to 5.5 using DIPEA. 0.5 mL aqueous solution of 0.015 g (0.078 mmol) EDC was added dropwise and stirred for 30 min with ice cooling. pH was raised to 8 using DIPEA and reacted for 40 min at room temperature. The end point of the reaction was monitored using HPLC and mass spectroscopy. The product was freeze-dried and purified by HPLC and obtained at 17.2 min peak (0.015 g, 52%).

¹H NMR (300 MHz, D₂O, ppm): δ 1.24 (s, —NHBoc), 3.75 (s, —COCH₃).

Mass spectrum (ESI+, turbospray), m/z 504 (M+H)⁺.

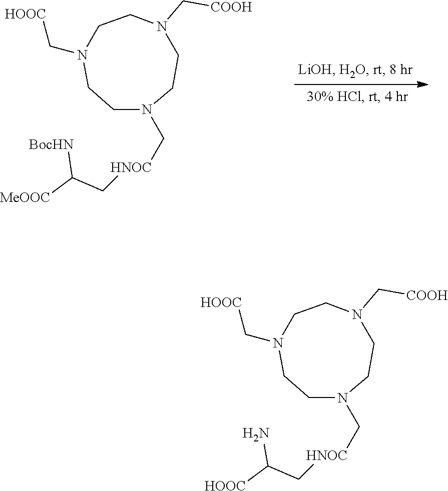

To the 0.5 mL aqueous solution of the above compound (0.015 g), 0.1 mL aqueous solution of lithium hydroxide (0.003 g, 0.125 mmol) was slowly added and hydrolyzed for 8 hr at room temperature. After checking the reaction finishing using HPLC and mass spectroscopy, pH was reduced to 1 using 30% HCl. The reaction mixture was purified using HPLC (0.010 g, 75%).

¹H NMR (300 MHz, D₂O, ppm): δ 3.09 (br, 4H), 3.24 (br, 4H), 3.35 (sbr, 4H), 3.59-3.64 (m, H), 3.68 (sbr, 2H), 3.87 (s, 4H), 4.08 (t, 1H).

¹³C NMR (80 MHz, D₂O, ppm): δ 39.7, 50.5, 50.9, 51.7, 53.7, 57.5, 58.4, 170.7, 172.1, 173.4.

Mass spectrum (ESI+, turbospray), m/z 390 (M+H)⁺.

### Example 4

### Synthesis of DOTA-ala

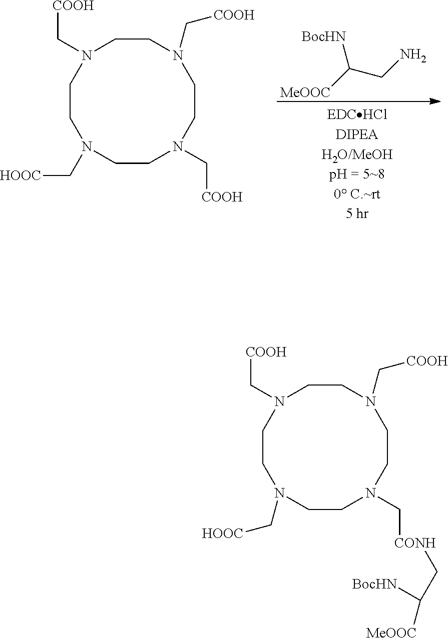

Protected aminoalanine (0.015 g, 0.068 mmol) solution in methanol (0.2 mL) was slowly added to 1.5 mL aqueous solution of 0.05 g (0.12 mmol) DOTA. The mixture was cooled by ice and pH was adjusted to 5 using DIPEA. 0.5 mL aqueous solution of 0.015 g (0.078 mmol) EDC was added dropwise and stirred for 20 min with ice cooling. PH was raised to 8 using DIPEA and reacted for 30 min at room temperature. The end point of the reaction was monitored using HPLC and mass spectroscopy. The product was freeze-dried and purified by HPLC and obtained at 17.2 min peak (0.018 g, 49%).

¹H NMR (300 MHz, D₂O, ppm): δ 1.25 (s, —NHBoc), 3.61 (s, —COCH₃).

Mass spectrum (ESI+, turbospray), m/z 605 (M+H)⁺.

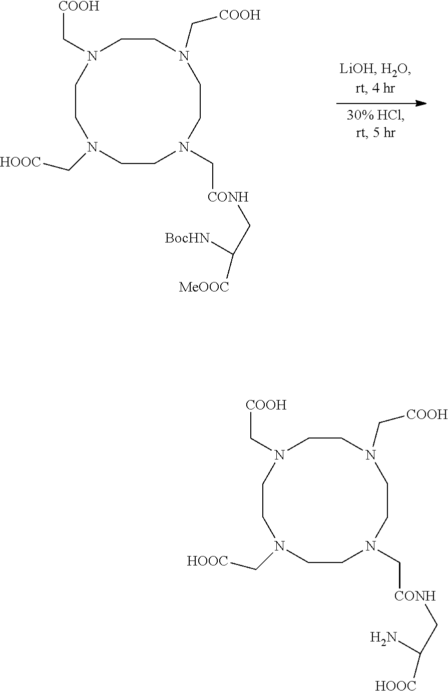

To the 0.5 mL aqueous solution of the above obtained compound (0.018 g), 0.1 mL aqueous solution of lithium hydroxide (0.002 g, 0.083 mmol) was slowly added and hydrolyzed for 4 hr at room temperature. After checking the reaction finishing using HPLC and mass spectroscopy, pH was reduced to 1 using 30% HCl. The reaction mixture was purified using HPLC (0.010 g, 75%).

¹H NMR (300 MHz, CDCl₃, ppm): δ 2.9-3.7 (br, 26H), 3.94-3.96 (q, —CH).

¹³C NMR (80 MHz, D₂O, ppm): δ 40.3, 45.5, 48.7, 49.2, 49.5, 49.8, 54.8, 167.5, 169.5, 178.1.

Mass spectrum (ESI+, turbospray), m/z 525 (M+H)⁺.

### Example 5

### Synthesis of NOTA-lys

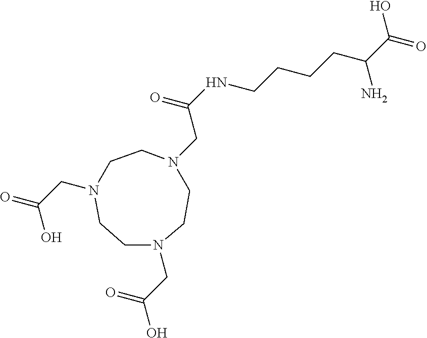

30% 4-dimethylaminopyridine (DMAP) solution in t-butanol was added to 2.5 mL t-butanol solution of tBoc anhydride (0.4 g, 1.84 mmol) and 0.5 g (1.31 mmol) (S)-tBoc-lys(Bz)-OH. The mixture was reacted for 9 hr with stirring, evaporated under reduced pressure, purified by column chromatography using ethylacetate/hexane=3:7 solution, and obtained (S)-tBoc-lys(Bz)-OtBu. Pd—C (10%) was added to 2.5 mL ethanol solution of (S)-tBoc-lys(Bz)-OtBu (0.4 g, 0.92 mmol), reacted for 3 hr with stirring under 1 atm hydrogen at room temperature, and filtered. The filter was washed with methylene chloride and the filtrate was evaporated under vacuum to give oil form (S)-ε-amino-tBoc-lys-OtBu. Acetonitrile (3 mL) solution of (S)-ε-amino-tBoc-lys-OtBu (0.06 g, 0.2 mmol) was added to 3 mL aqueous solution of NOTA (0.1 g, 0.329 mmol) and HOBT (0.027 g, 0.2 mmol) mixture. DCC (0.054 g, 0.26 mmol) in 0.1 mL pyridine was added to the mixture and stirred for 15 hr at room temperature. The reaction mixture was filtered, the filtrate was evaporated under reduced pressure, and the product was purified by RP-HPLC [10 mM HCl solution (A)/acetonitrile (B); 100% A 5 min, 0 to 70% B 25 min]. After evaporation under reduced pressure, the resulting residue was dissolved in 0.2 mL water and reacted with 4 M HCl in dioxane solution (3 mL) for 4 hr at room temperature. After evaporation under reduced pressure, the resulting residue was purified by RP-HPLC [0 to 40% B 20 min]. Mass spectrum (ESI+), m/z 432.2 (M+H)⁺.

### Example 6

### Synthesis of DOTA-lys

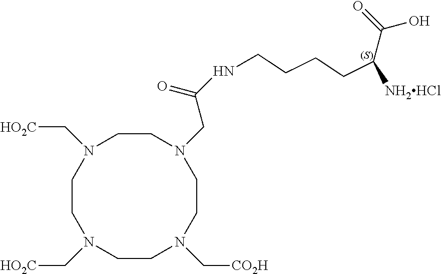

Acetonitrile (3 mL) solution of (S)-ε-amino-tBoc-lys-OtBu (0.05 g, 0.15 mmol) was added to 2.5 mL aqueous solution of DOTA (0.1 g, 0.3 mmol) and HOBT (0.02 g, 0.15 mmol) mixture. DCC (0.041 g, 0.3 mmol) in 0.1 mL pyridine was added to the mixture and stirred for 15 hr at room temperature. The reaction mixture was filtered, the filtrate was evaporated under reduced pressure, and the product was purified by RP-HPLC [10 mM HCl solution (A)/acetonitrile (B); 100% A 5 min, 0 to 70% B 25 min]. After evaporation under reduced pressure, the resulting residue 0.022 g was dissolved in 0.2 mL water and reacted with 4 M HCl in dioxane solution (3 mL) for 3 hr at room temperature. After evaporation under reduced pressure, the resulting residue was purified by RP-HPLC [0 to 40% B 20 min]. Mass spectrum (ESI+), m/z 533.5 (M+H)⁺.

### Example 7

### Synthesis of NOTA-homoser

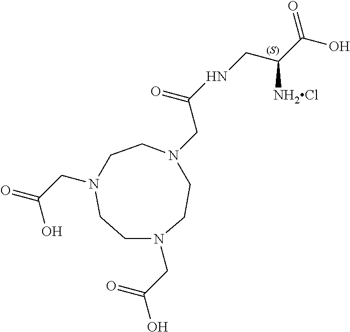

Acetonitrile (3 mL) solution of (S)-γ-amino-N-tBoc-homoser-OtBu (0.025 g, 0.08 mmol) was added to 2.5 mL aqueous solution of NOTA (0.05 g, 0.16 mmol) and HOBT (0.012 g, 0.08 mmol) mixture. DCC (0.025 g, 0.16 mmol) in 0.1 mL pyridine was added to the mixture and stirred for 15 hr at room temperature. The reaction mixture was filtered, the filtrate was evaporated under reduced pressure, and the product was purified by RP-HPLC [10 mM HCl solution (A)/acetonitrile (B); 100% A 5 min, 0 to 70% B 25 min]. After evaporation under reduced pressure, the resulting residue 0.01 g was dissolved in 1 mL water and reacted with 4 M HCl in dioxane solution (4 mL) for 6 hr at room temperature. After evaporation under reduced pressure, the resulting residue was purified by RP-HPLC [0 to 40% B 20 min]. Mass spectrum (ESI+), m/z 404.2 (M+H)⁺.

### Example 8

### Synthesis of DOTA-homoser

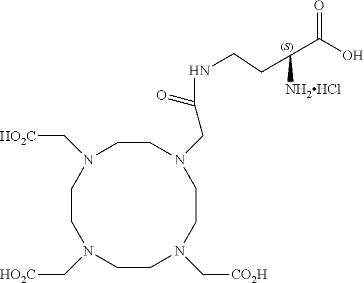

Acetonitrile (2.5 mL) solution of (S)-γ-amino-N-tBoc-homoser-OtBu (0.038 g, 0.25 mmol) was added to 2.5 mL aqueous solution of DOTA (0.1 g, 0.49 mmol) and HOBT (0.033 g, 0.25 mmol) mixture. DCC (0.051 g, 0.25 mmol) in 0.1 mL pyridine was added to the mixture and stirred for 15 hr at room temperature. The reaction mixture was filtered, the filtrate was evaporated under reduced pressure, and the product was purified by RP-HPLC [10 mM HCl solution (A)/acetonitrile (B); 100% A 5 min, 0 to 70% B 25 min]. After evaporation under reduced pressure, the resulting residue was dissolved in 0.5 mL water and reacted with 4 M HCl in dioxane solution (2.5 mL) for 8 hr at room temperature. After evaporation under reduced pressure, the resulting residue was purified by RP-HPLC [0 to 40% B 20 min] to obtain HCl salt form of the product. Mass spectrum (ESI+), m/z 505.2 (M+H)⁺.

### Example 9

### Synthesis of Ga-DO2A-ala

5 mL DO2a-ala (0.12 g, 0.32 mmol) in 1 M sodium acetate buffer (pH 4.0) was added to 7 mL GaCl₃ (0.056 g, 0.32 mmol) solution in the same buffer and heated for 10 min at 100° C. The reaction mixture was filtered by PVDF filter (0.45 μm), purified by HPLC and collected a peak at 6.5 min.

Mass spectrum (ESI+, turbospray), m/z 442.1 (M+H)⁺.

### Example 10

### Synthesis of Ga-DO3A-ala

2.5 mL DO3A-ala (0.03 g, 0.07 mmol) in 1 M sodium acetate buffer (pH 4.0) was added to 3 mL GaCl₃ (0.012 g, 0.07 mmol) solution in the same buffer and heated for 10 min at 100° C. The reaction mixture was filtered by PVDF filter (0.45 μm), purified by HPLC and collected a peak at 6.3 min.

Mass spectrum (ESI+, turbospray), m/z 500.1 (M+H)⁺.

### Example 11

### Synthesis of Ga-NOTA-ala

HCl salt form of NOTA-ala (15 mg, 0.038 mmol) was dissolved in 0.4 mL distilled water and pH was adjusted to 5 using 0.1 M HCl and 0.5 M sodium phosphate buffer. 0.2 mL GaCl₃ (0.038 mmol) solution was added dropwise and heated for 10 min at 100° C. The reaction mixture was filtered by PVDF filter (0.45 μm), purified by HPLC and collected a peak at 6.3 min.

¹H NMR (300 MHz, D₂O, pH˜4, ppm): δ 4.22 (t, 1H, J=3.8 Hz), 4.05 (m, 2H), 3.77 (s, 2H), 3.70 (s, 4H), 3.60-3.21 (br, 8H), 3.20-2.95 (br, 4H).

¹³C NMR (80 MHz, D₂O, ppm): δ 39.2, 42.1, 54.0, 54.1, 62.5, 62.6, 175.2, 175.7, 175.9.

Mass spectrum (ESI+, turbospray), m/z 456.1 (M+H)⁺.

### Example 12

### Synthesis of Ga-DOTA-ala

HCl salt form of DOTA-ala (12 mg, 0.031 mmol) was dissolved in 0.5 mL distilled water and 0.2 mL GaCl₃ (27 mg, 0.153 mmol) solution was added dropwise and heated for 10 min at 100° C. The reaction mixture was filtered by PVDF filter (0.45 μm), purified by HPLC and collected a peak at 6.3 min.

Mass spectrum (ESI+, turbospray), m/z 595 (M+H)⁺.

### Example 13

### ⁶⁸Ga Labeling of NOTA-ala, DOTA-ala, DO2A-ala, and DO3A-ala

⁶⁸GaCl₃ was obtained from ⁶⁸Ge/⁶⁸Ga-generator by elution with 0.1 M HCl. Each ligand (0.016 μmol) was mixed with 0.1 mL sodium acetate buffer solution (pH 3.5) and 1 mL of ⁶⁸GaCl₃ solution in 0.1 M HCl and heated for 10 min in boiling water bath. Labeling efficiencies were measure by instant thin layer chromatography-silica gel (ITLC-SG, Pall Life Sciences, New York, U.S.A.) using 0.1 M sodium carbonate as a elution solvent. Free ⁶⁸Ga remained at the origin and labeled ⁶⁸Ga moved to Rf=9˜1.0.

### Example 14

### ¹¹¹In Labeling of DOTA-ala, DOTA-homoala, DOTA-lys, DO2A-ala, DO3A-ala, and DO3A-homoala

¹¹¹InCl₃ was purchased from Perkin Elmer (Waltham, Mass.). Each ligand (0.020 nmol) was mixed with 7.4 MBq of ¹¹¹InCl₃ and 0.4 mL of 0.1 M sodium acetate buffer solution (pH 4.0). Mixtures were vortexed for 20 sec and then incubated for 10 min at 95° C. Labeling efficiencies were measure by instant thin layer chromatography-silica gel (ITLC-SG, Pall Life Sciences, New York, U.S.A.) using 0.1 M sodium carbonate as a elution solvent. Free ¹¹¹In remained at the origin and labeled ¹¹¹In moved to Rf=0.9˜1.0.

### Preparation of Control. ⁶⁸Ga Labeling of NOTA, DOTA, DO2A, and DO3A.

⁶⁸GaCl₃ was obtained from ⁶⁸Ge/⁶⁸Ga-generator by elution with 0.1 M HCl. Each ligand (0.016 μmol) was mixed with 0.1 mL sodium acetate buffer solution (pH 3.5) and 1 mL of ⁶⁸GaCl₃ solution in 0.1 M HCl and heated for 10 min in boiling water bath. Labeling efficiencies were measure by instant thin layer chromatography-silica gel (ITLC-SG, Pall Life Sciences, New York, U.S.A.) using 0.1 M sodium carbonate as a elution solvent. Free ⁶⁸Ga remained at the origin and labeled ⁶⁸Ga moved to Rf=0.9˜1.0.

### Experiment 1. In Vitro Cell Uptake

Human colon cancer cell line SNU-C4 was purchased from Korea Cell Line Bank (KCLB). Human thyroid cell line ARO, human liver cancer cell line Hep3B, human glioma cell line U251MG and U87MG, and mouse colon cell line CT-26 were purchased from American Type Culture Collection (ATCC). SNU-C4 and ARO were culture in RPMI1640 (Welgene Inc., Korea) and Hep3B, U251 MG, U87MG, and CT-26 were cultured in DMEM (Welgene Inc., Korea). Penicillin, streptomycin and amphotericin B (10,000 IU/mL, 10 mg/mL and 25 μg/mL, respectively, Mediatech Inc., U.S.A.) 1% mixture and fetal bovine serum (Welgene Inc., Korea) were added to all cell culture media. Cell culture was done in 37° C. incubator with supply of 5% CO₂.

About 1.8×10⁵ cells/mL CT-26 and 1.2×10⁵ cells/mL U87MG were put into 24-well incubation plate and incubated for 20 hr. When the each well was about 80% confluent with cells, 296 kBq/0.5 mL of ⁶⁸Ga labeled agent was added to each well. Culture supernatants were discarded at each time point and cells were washed with ice-cooled Hank's balanced salt solution (HBSS, pH7.3, Gibco, U.S.A.) 2 times. The cells were dissolved by 0.5% sodium dodecylsulfate (SDS), recovered and counted using a gamma counter (Packard, Can berra Co., U.S.A.). Total protein amount in each sample was measured by bicinchonic acid (BCA, Pierce, U.S.A.) method.

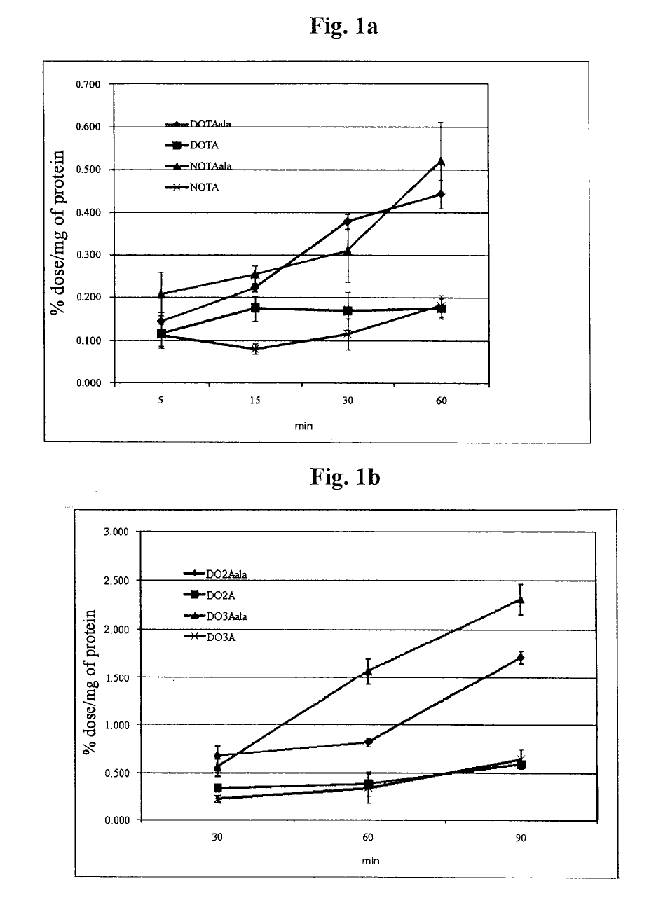

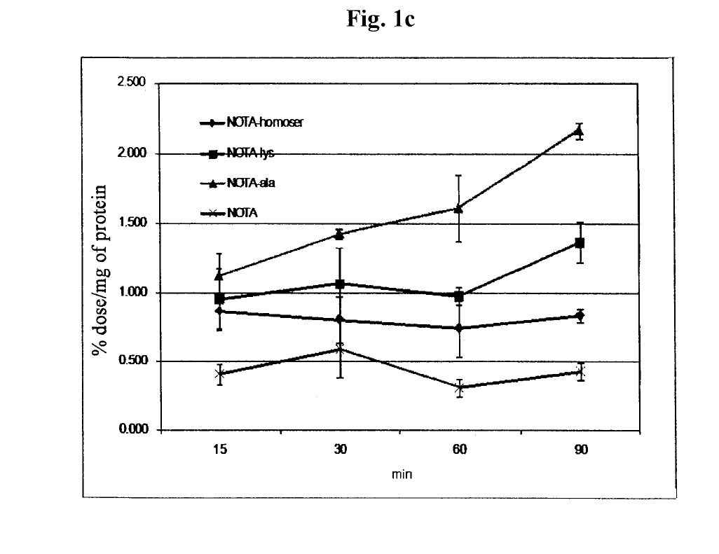

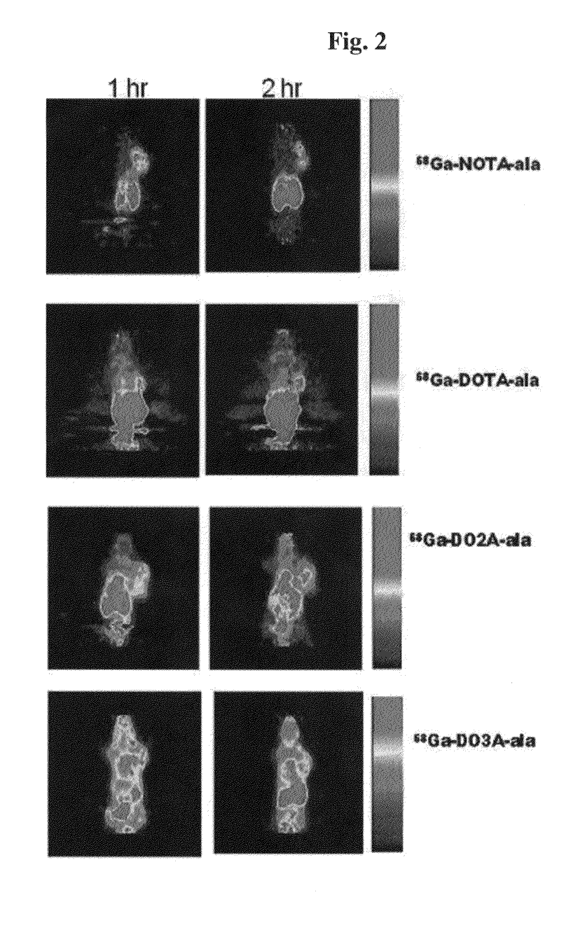

According to the cell uptake experiment, ⁶⁸Ga-DOTA-ala, ⁶⁸Ga-NOTA-ala, ⁶⁸Ga-DO2A-ala, ⁶⁸Ga-DO3A-ala, ⁶⁸Ga-NOTA-ala, ⁶⁸Ga-NOTA-homoser, and ⁶⁸Ga-NOTA-lys of the present invention showed significantly increase tumor cell uptakes than control agents ⁶⁸Ga-DOTA, ⁶⁸Ga-NOTA, ⁶⁸Ga-DO2A, and ⁶⁸Ga-DO3A in FIGS. 1a, 1b and 1c.

### Experiment 2. Biodistribution Studies in Tumor Xenografted Mice

Human colon cancer cell line SNU-C4 was cultured in RPMI1640 media containing 10% fetal bovine serum and harvested using trypsin. The cells were washed using 10 mL PBS and centrifugation at 3,000 rpm. Each nude mouse was injected with 2×10⁵ cells/0.1 mL on the right shoulder subcutaneously. On 13 days post-injection, each 10 μCi/0.1 mL of ⁶⁸Ga labeled agent was injected into the xenografted mice through the tail vein. The injected mice were sacrificed at 10 min, 30 min, 1 hr, and 2 hr, cancer, blood, muscle, heart, liver, spleen, stomach, intestine, and bone were obtained, weighed, and their radioactivities were counted. The results were expressed as percentages of injected dose per gram tissue (% ID/g).

Table 1 shows that ⁶⁸Ga-NOTA-ala tumor uptake was higher and increasing by time than that of ⁶⁸Ga-NOTA: 1.07 times at 10 min, 1.09 times at 30 min, 1.24 times at 1 hr, and 1.42 times at 2 hr. Table 2 shows that ⁶⁸Ga-DOTA-ala tumor uptake was higher than that of ⁶⁸Ga-DOTA at all time point, and the difference was the highest especially at 30 min. Table 3 also shows ⁶⁸Ga-DO2A-ala tumor uptake was always higher than that of ⁶⁸Ga-DO2A and the difference was the highest at 30 min. Table 4 shows that ⁶⁸Ga-DO3A-ala tumor uptake was higher and increasing by time than that of ⁶⁸Ga-DO3A, and showed higher differences than the other agents. In conclusion, ⁶⁸Ga labeled heterocycle-amino acids of the present invention showed higher tumor uptakes than the control heterocyclic compounds, and thus proved the feasibility of using them as cancer imaging radiopharmaceuticals.

TABLE 1
a. Biodistribution study of ⁶⁸Ga-NOTA in SNU-C4
xenografted nude mice after intravenous injection.

| | ⁶⁸Ga-NOTA | | | |
|---|---|---|---|---|
| % ID/g | 10 min (n = 4) | 30 min (n = 3) | 1 hr (n = 4) | 2 hr (n = 4) |
| Blood | 7.13 ± 0.77 | 2.55 ± 0.31 | 0.41 ± 0.24 | 0.07 ± 0.00 |
| Muscle | 1.68 ± 0.86 | 0.77 ± 0.19 | 0.20 ± 0.17 | 0.04 ± 0.01 |
| Heart | 1.83 ± 0.16 | 0.59 ± 0.07 | 0.15 ± 0.10 | 0.07 ± 0.07 |
| Liver | 1.98 ± 0.19 | 1.44 ± 0.29 | 1.04 ± 0.35 | 1.21 ± 0.21 |
| Spleen | 1.41 ± 0.19 | 0.62 ± 0.09 | 0.22 ± 0.09 | 0.19 ± 0.03 |
| Stomach | 2.13 ± 0.26 | 0.76 ± 0.31 | 0.20 ± 0.10 | 0.11 ± 0.03 |
| Intestine | 1.52 ± 0.08 | 0.84 ± 0.10 | 0.33 ± 0.14 | 0.47 ± 0.17 |
| Kidney | 11.61 ± 0.61 | 7.44 ± 1.29 | 3.56 ± 2.10 | 2.73 ± 0.32 |
| Bone | 1.95 ± 0.06 | 0.65 ± 0.16 | 0.15 ± 0.09 | 0.06 ± 0.07 |
| Tumor | 2.69 ± 0.19 | 1.28 ± 0.22 | 0.59 ± 0.18 | 0.43 ± 0.06 |

b. Biodistribution study of ⁶⁸Ga-NOTA-ala in SNU-C4
xenografted nude mice after intravenous injection.

| | ⁶⁸Ga-NOTA-ala | | | |
|---|---|---|---|---|
| % ID/g | 10 min (n = 4) | 30 min (n = 4) | 1 hr (n = 4) | 2 hr (n = 4) |
| Blood | 7.46 ± 0.78 | 2.69 ± 0.59 | 0.73 ± 0.05 | 0.15 ± 0.03 |
| Muscle | 2.29 ± 0.52 | 0.87 ± 0.33 | 0.26 ± 0.05 | 0.06 ± 0.04 |
| Heart | 2.13 ± 0.13 | 0.71 ± 0.17 | 0.26 ± 0.03 | 0.10 ± 0.05 |
| Liver | 2.08 ± 0.25 | 1.34 ± 0.22 | 1.05 ± 0.04 | 0.84 ± 0.18 |
| Spleen | 1.84 ± 0.10 | 0.93 ± 0.14 | 0.55 ± 0.07 | 0.48 ± 0.11 |
| Stomach | 2.46 ± 0.31 | 0.79 ± 0.17 | 0.33 ± 0.08 | 0.18 ± 0.05 |
| Intestine | 1.74 ± 0.22 | 0.77 ± 0.19 | 0.44 ± 0.07 | 0.35 ± 0.11 |
| Kidney | 20.96 ± 7.35 | 9.07 ± 1.68 | 5.81 ± 0.57 | 4.04 ± 0.63 |
| Bone | 2.22 ± 0.34 | 1.05 ± 0.35 | 0.47 ± 0.09 | 0.10 ± 0.07 |
| Tumor | 2.87 ± 0.58 | 1.40 ± 0.43 | 0.73 ± 0.25 | 0.61 ± 0.34 |

TABLE 2
a. Biodistribution study of ⁶⁸Ga-DOTA in SNU-C4
xenografted nude mice after intravenous injection.

| | ⁶⁸Ga-DOTA | | | |
|---|---|---|---|---|
| % ID/g | 10 min (n = 4) | 30 min (n = 4) | 1 hr (n = 4) | 2 hr (n = 4) |
| Blood | 7.52 ± 0.77 | 2.76 ± 0.41 | 0.90 ± 0.19 | 0.21 ± 0.01 |
| Muscle | 2.08 ± 0.11 | 0.70 ± 0.12 | 0.26 ± 0.12 | 0.10 ± 0.04 |
| Heart | 2.04 ± 0.19 | 0.68 ± 0.09 | 0.23 ± 0.08 | 0.08 ± 0.05 |
| Liver | 1.30 ± 0.15 | 0.60 ± 0.08 | 0.32 ± 0.04 | 0.21 ± 0.03 |
| Spleen | 1.41 ± 0.08 | 0.56 ± 0.07 | 0.27 ± 0.06 | 0.18 ± 0.06 |
| Stomach | 1.86 ± 0.39 | 0.72 ± 0.15 | 0.26 ± 0.06 | 0.10 ± 0.03 |
| Intestine | 1.62 ± 0.11 | 0.68 ± 0.08 | 0.36 ± 0.05 | 0.26 ± 0.08 |
| Kidney | 16.19 ± 1.83 | 6.33 ± 0.95 | 4.13 ± 1.13 | 2.92 ± 0.97 |
| Bone | 1.86 ± 0.22 | 0.85 ± 0.11 | 0.33 ± 0.10 | 0.17 ± 0.17 |
| Tumor | 2.55 ± 0.81 | 1.26 ± 0.23 | 0.60 ± 0.06 | 0.41 ± 0.09 |

b. Biodistribution study of ⁶⁸Ga-DOTA-ala in SNU-C4
xenografted nude mice after intravenous injection

| | ⁶⁸Ga-DOTA-ala | | | |
|---|---|---|---|---|
| % ID/g | 10 min (n = 4) | 30 min (n = 4) | 1 hr (n = 4) | 2 hr (n = 7) |
| Blood | 7.87 ± 1.11 | 2.63 ± 0.11 | 1.30 ± 0.28 | 0.65 ± 0.11 |
| Muscle | 2.12 ± 0.64 | 0.90 ± 0.11 | 0.47 ± 0.16 | 0.14 ± 0.03 |
| Heart | 2.19 ± 0.47 | 0.78 ± 0.07 | 0.43 ± 0.10 | 0.15 ± 0.05 |
| Liver | 1.57 ± 0.22 | 0.80 ± 0.12 | 0.56 ± 0.06 | 0.39 ± 0.10 |
| Spleen | 1.64 ± 0.26 | 0.74 ± 0.10 | 0.47 ± 0.04 | 0.33 ± 0.08 |
| Stomach | 2.63 ± 0.63 | 0.90 ± 0.17 | 0.45 ± 0.10 | 0.21 ± 0.05 |
| Intestine | 1.91 ± 0.48 | 0.74 ± 0.11 | 0.52 ± 0.10 | 0.40 ± 0.10 |
| Kidney | 50.94 ± 18.62 | 44.79 ± 11.99 | 67.43 ± 16.10 | 48.79 ± 20.36 |
| Bone | 2.52 ± 0.64 | 1.20 ± 0.17 | 0.90 ± 0.12 | 0.70 ± 0.26 |
| Tumor | 3.33 ± 0.58 | 2.23 ± 0.64 | 0.93 ± 0.22 | 0.66 ± 0.25 |

TABLE 3

| | | | | |
|---|---|---|---|---|
| % ID/g | 10 min (n = 4) | 30 min (n = 4) | 1 hr (n = 4) | 2 hr (n = 4) |

a. Biodistribution study of ⁶⁸Ga-DO2A in SNU-C4
xenografted nude mice after intravenous injection.

| | ⁶⁸Ga-DO2A | | | |
|---|---|---|---|---|
| Blood | 7.82 ± 1.05 | 3.61 ± 0.74 | 1.12 ± 0.27 | 0.44 ± 0.06 |
| Muscle | 2.81 ± 0.82 | 1.45 ± 0.42 | 0.45 ± 0.17 | 0.13 ± 0.07 |
| Heart | 2.33 ± 0.32 | 0.97 ± 0.20 | 0.37 ± 0.10 | 0.23 ± 0.03 |
| Lung | 6.96 ± 1.06 | 3.57 ± 0.80 | 1.32 ± 0.35 | 0.87 ± 0.25 |
| Liver | 2.91 ± 0.31 | 2.75 ± 0.49 | 2.31 ± 0.27 | 2.17 ± 0.20 |
| Spl- | 1.86 ± 0.24 | 1.03 ± 0.22 | 0.47 ± 0.10 | 0.38 ± 0.08 |
| een | | | | |
| Stom- | 2.52 ± 0.46 | 1.22 ± 0.32 | 0.38 ± 0.05 | 0.18 ± 0.03 |
| ach | | | | |
| Intes- | 2.24 ± 0.36 | 1.27 ± 0.25 | 0.68 ± 0.11 | 0.62 ± 0.07 |
| tine | | | | |
| Kidney | 32.32 ± 21.63 | 13.61 ± 3.98 | 8.46 ± 2.11 | 6.90 ± 1.10 |
| Bone | 2.56 ± 0.61 | 1.54 ± 0.53 | 0.72 ± 0.23 | 0.58 ± 0.19 |
| Tumor | 2.71 ± 0.51 | 2.10 ± 0.23 | 0.99 ± 0.10 | 0.67 ± 0.12 |

b. Biodistribution study of ⁶⁸Ga-DO2A-ala in SNU-C4
xenografted nude mice after intravenous injection.

| | ⁶⁸Ga-DO2A-Ala | | | |
|---|---|---|---|---|
| Blood | 7.75 ± 0.97 | 5.26 ± 1.36 | 3.12 ± 0.89 | 1.94 ± 0.24 |
| Muscle | 2.08 ± 0.43 | 1.81 ± 0.77 | 0.87 ± 0.40 | 0.49 ± 0.08 |
| Heart | 1.98 ± 0.06 | 1.19 ± 0.34 | 0.79 ± 0.34 | 0.45 ± 0.10 |
| Lung | 8.45 ± 0.98 | 8.23 ± 2.41 | 4.21 ± 1.47 | 3.23 ± 0.49 |
| Liver | 1.74 ± 0.25 | 1.46 ± 0.27 | 1.39 ± 0.47 | 0.99 ± 0.04 |
| Spl- | 1.61 ± 0.26 | 1.30 ± 0.29 | 1.24 ± 0.34 | 0.82 ± 0.09 |
| een | | | | |
| Stom- | 2.48 ± 0.62 | 1.49 ± 0.62 | 12.82 ± 7.46 | 0.38 ± 0.05 |
| ach | | | | |
| Intes- | 1.91 ± 0.33 | 1.40 ± 0.38 | 3.91 ± 2.96 | 0.77 ± 0.03 |
| tine | | | | |
| Kidney | 13.33 ± 4.30 | 14.50 ± 8.89 | 4.71 ± 2.26 | 3.99 ± 0.54 |
| Bone | 2.60 ± 0.56 | 2.22 ± 0.49 | 1.57 ± 0.52 | 1.06 ± 0.52 |
| Tumor | 2.88 ± 0.39 | 2.30 ± 0.53 | 1.05 ± 0.33 | 1.08 ± 0.18 |

TABLE 4

| | | | | |
|---|---|---|---|---|
| | | 30 min | | |
| % ID/g | 10 min (n = 3) | (n = 4) | 1 hr (n = 4) | 2 hr (n = 4) |

a. Biodistribution study of ⁶⁸Ga-DO3A in SNU-C4
xenografted nude mice after intravenous injection.

| | ⁶⁸Ga-DO3A | | | |
|---|---|---|---|---|
| Blood | 6.96 ± 0.18 | 3.21 ± 0.58 | 1.03 ± 0.34 | 0.22 ± 0.02 |
| Muscle | 1.69 ± 0.39 | 0.92 ± 0.27 | 0.32 ± 0.17 | 0.10 ± 0.03 |
| Heart | 1.75 ± 0.12 | 0.77 ± 0.11 | 0.32 ± 0.09 | 0.06 ± 0.09 |
| Lung | 5.81 ± 0.54 | 2.82 ± 0.59 | 1.26 ± 0.28 | 0.61 ± 0.23 |
| Liver | 1.99 ± 0.03 | 1.35 ± 0.13 | 1.02 ± 0.16 | 0.93 ± 0.05 |
| Spleen | 1.63 ± 0.08 | 0.95 ± 0.14 | 0.64 ± 0.09 | 0.53 ± 0.11 |
| Stomach | 2.37 ± 0.56 | 1.00 ± 0.24 | 0.43 ± 0.20 | 0.16 ± 0.05 |
| Intestine | 1.87 ± 0.06 | 1.00 ± 0.14 | 0.64 ± 0.17 | 0.42 ± 0.08 |
| Kidney | 11.96 ± 0.51 | 7.85 ± 1.00 | 6.52 ± 3.13 | 7.50 ± 3.82 |
| Bone | 1.64 ± 0.48 | 0.97 ± 0.28 | 0.38 ± 0.12 | 0.28 ± 0.33 |
| Tumor | 3.14 ± 0.73 | 1.83 ± 0.20 | 0.93 ± 0.33 | 0.54 ± 0.04 |

b. Biodistribution study of ⁶⁸Ga-DO3A-ala in SNU-C4
xenografted nude mice after intravenous injection.

| | ⁶⁸Ga-DO3A-Ala | | | |
|---|---|---|---|---|
| Blood | 11.12 ± 0.49 | 8.35 ± 0.29 | 7.97 ± 1.29 | 6.07 ± 1.13 |
| Muscle | 3.52 ± 0.42 | 2.03 ± 0.41 | 1.38 ± 0.39 | 1.33 ± 0.48 |
| Heart | 2.70 ± 0.18 | 1.80 ± 0.22 | 1.69 ± 0.40 | 1.30 ± 0.40 |
| Lung | 13.60 ± 2.37 | 9.35 ± 1.00 | 8.40 ± 1.17 | 8.04 ± 0.83 |
| Liver | 2.58 ± 0.07 | 2.30 ± 0.13 | 2.35 ± 0.25 | 2.52 ± 0.41 |
| Spleen | 2.59 ± 0.30 | 2.33 ± 0.15 | 2.33 ± 0.44 | 2.19 ± 0.35 |
| Stomach | 1.97 ± 0.18 | 1.53 ± 0.17 | 1.53 ± 0.37 | 11.42 ± 5.04 |
| Intestine | 2.48 ± 0.13 | 1.74 ± 0.14 | 1.87 ± 0.33 | 4.38 ± 1.14 |
| Kidney | 60.06 ± 14.07 | 9.10 ± 2.19 | 4.82 ± 0.99 | 6.06 ± 2.27 |
| Bone | 4.06 ± 0.44 | 3.63 ± 0.49 | 4.73 ± 0.69 | 5.00 ± 2.41 |
| Tumor | 3.22 ± 0.90 | 2.49 ± 0.40 | 2.44 ± 0.46 | 2.98 ± 0.58 |

### Experiment 3. Pet Imaging of Tumor Xenografted Mice

Human colon cancer cell line SNU-C4 was cultured in RPMI1640 media containing 10% fetal bovine serum and harvested using trypsin. The cells were washed using 10 mL PBS and centrifugation at 3,000 rpm. Each nude mouse was injected with 2×10⁵ cells/0.1 mL on the right shoulder subcutaneously. On 14 days post-injection, each 10 μCi/0.1 mL of ⁶⁸Ga labeled agent was injected into the xenografted mice through the tail vein. The injected mice were anesthetized using 2% isoflurane, PET images were obtained at 1 and 2 hr post-injection using rodent R4, microPET scanner (Concorde Microsystem Inc.). The images were analyzed using ASIPro software (Concorde Microsystem Inc.).

According to the results, all the examined heterocycle-amino acids, ⁶⁸Ga-NOTA-ala, ⁶⁸Ga-DOTA-ala, ⁶⁸Ga-DO2A-ala, and ⁶⁸Ga-DO3A-ala showed high tumor uptakes, especially ⁶⁸Ga-NOTA-ala showed the highest quality image.

### INDUSTRIAL APPLICABILITY

As apparent from the above description, the present invention provides novel heterocycle-amino acid derivatives or pharmaceutically acceptable salts thereof, and radioactive or non-radioactive metal complexes thereof. The present invention also provides a composition for imaging cancer comprising a complex of heterocycle-amino acid derivative, which can be prepared easily and is characterized by its high rate of accumulation in cancer tissue, thereby being capable of achieving an efficient cancer image.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A compound of Formula (1) or a pharmaceutically acceptable salt thereof: 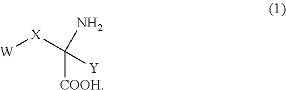
wherein W is 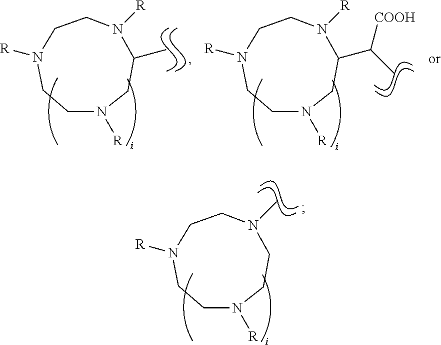
R is independently H, C₁ to C₆ alkyl, or —(CH₂)ₐCOOH; a and i are independently integer 1 to 6; X is repeatedly or non-repeatedly linked structure composed of 1 to 6 residue(s) of one or more selected from the group consisting of —CH₂—, —NH—, —O—, —S—, —CS—, and —CO—; and Y is H or a methyl residue.

2. The compound or a pharmaceutically acceptable salt thereof according to claim 1, wherein W is 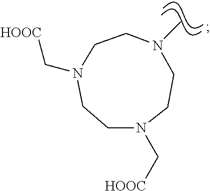
X is 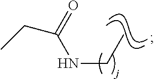
and j is an integer 1 to 6.

3. The compound or a pharmaceutically acceptable salt thereof according to claim 1, wherein W is 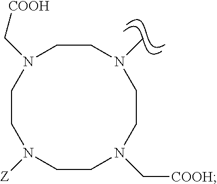
X is 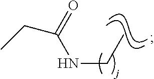
j is an integer 1 to 6; and Z is H or —CH₂COOH.

4. The compound or a pharmaceutically acceptable salt thereof according to claim 1, wherein X is CH₂ₖ; and k is an integer 1 to 6.

5. The compound or a pharmaceutically acceptable salt thereof according to claim 1, wherein amino acid arrangement is L type isomer.

6. The compound or a pharmaceutically acceptable salt thereof according to claim 1, wherein amino acid arrangement is D type isomer.

7. A complex of radioactive or non-radioactive metals with the compound or a pharmaceutically acceptable salt thereof according to claim 1.

8. The complex according to claim 7, wherein radioactive or non-radioactive metals are selected from the group consisting of Cr, Fe, Co, Ni, Cu, Ga, Sr, Y, Zr, Mo, Tc, Ru, Rh, Pd, Cd, In, Sn, Ba, La, Sm, Gd, Dy, Ho, Lu, Re, Ir, Pb, and Bi.

9. The complex according to claim 7, wherein radioactive metals are selected from the group consisting of ¹¹¹In, ⁶⁸Ga, ⁶⁷Ga, ⁶⁰Cu, ⁶¹Cu, ⁶²Cu, ⁶⁴Cu, ⁶⁷Cu, ⁸⁵Y, ⁸⁶Y, ⁸⁷Y, ⁹⁰Y, ¹⁷⁷Lu, ^{117m}Sn, ¹⁰³Pd, and ¹⁶⁶Ho.

10. A kit for the preparation of a sterile non-pyrogenic sealed vial of solution, frozen or lyophilized state containing 1 ng˜100 mg of the compound or a pharmaceutically acceptable salt thereof according to claim 1.
